(19) United States
(12) Patent Application Publication
(10) Pub. No.: US 2018/0251765 A1
(43) Pub. Date: Sep. 6, 2018

(54) HIGH-THROUGHPUT SPLIT APTAMER SCREENING ASSAY

(71) Applicant: BELLBROOK LABS, Madison, WI (US)

(72) Inventors: Meera Kumar, Madison, WI (US); Robert G. Lowery, Madison, WI (US)

(73) Assignee: Bellbrook Labs, Madison, WI (US)

(21) Appl. No.: 15/756,851

(22) PCT Filed: Sep. 7, 2016
(86) PCT No.: PCT/US2016/050564

§ 371 Date: Mar. 1, 2018

Related U.S. Application Data
(60) Provisional application No. 62/215,555, filed on Sep. 8, 2015.

(51) Int. Cl. C12N 15/115; C12Q 1/6804; C12Q 1/66; C12Q 1/6818

(52) U.S. Cl. CPC C12N 15/115; C12Q 1/6804; C12Q 1/66; C12Q 1/6818; C12Y 113/12; C12Q 2561/107; C12Q 2561/119; C12Q 2563/103; C12Q 2565/101; C12Q 2525/205

(57) ABSTRACT

Methods and materials for development of high-throughput screening assays using split aptamers are provided by this invention.

### BACKGROUND OF THE INVENTION

### Field of Invention

The present invention relates to methods and materials for development of high-throughput screening assays using split aptamers.

### Description of Related Art

Aptamers are nucleic acid affinity reagents that have been developed for detection of diverse ligands ranging in size from small molecules (cocaine, thalidomide, ATP, dopamine) to proteins (VEGF, EGFR) to cells (McKeague & Derosa, 2012, J Nucleic Acids 2012:748913; Yuce et al., 2015, Analyst 140(16):5379-99). In addition, naturally occurring aptamers, called riboswitches, have been discovered for diverse biomolecules including sugars, amino acids, and cyclic nucleotides (Breaker, 2012, Cold Spring Harb. Perspect. Biol. 4(2):a003566. DNA and RNA aptamers can exhibit subnanomolar affinity and exquisite selectivity for their ligands (McKeague & Derosa, 2012, J Nucleic Acids 2012:748913; Yuce et al., 2015, Analyst 140(16):5379-99) and are thus well suited for detecting analytes in complex mixtures like cell lysates or serum. Moreover, evidence suggests that aptamers are more specific than antibodies for small or structurally subtle epitopes such as methyl and acetyl moieties. For example, a well characterized RNA aptamer for theophylline discriminates against caffeine with more than 10⁴-fold selectivity on the basis of a single methyl group (McKeague & Derosa, 2012, J Nucleic Acids 2012:748913). Similarly, riboswitches can discriminate with more than 1000-fold selectivity on the basis of a single methyl group; e.g., for S-adenosylhomocysteine (SAH) versus S-adenosylmethionine (SAM) (Wang et al., 2008, Mol Cell. 29(6):691-702).

The most commonly used detection formats for HTS applications are time-resolved fluorescence energy transfer (TR-FRET), fluorescence polarization (FP), fluorescence index (FI), and luminescence (Jones et al., 2004, Assay Guidance Manual, Ed. Sittampalam et al.). Unfortunately, the vast majority of aptamer based assays developed have used detection formats that are not compatible with commonly placed HTS instrumentation (Famulok & Mayer, 2011, Acc. Chem. Res. 44(12):1349-58; Famulok & Mayer, 2014, Chem. Biol. 21(9):1055-8; Kim & Gu, 2014, Adv. Biochem. Eng. Biotechnol. 140:29-67. For example, many aptamer based assays use aptamers attached to nanoparticles like gold nanoparticles or carbon nanotubes and produce electrochemical signals that require specialized electrodes for detection or optical signals such as resonance light scattering that require highly specialized and expensive instruments, in some cases home-made (Iqbal et al., 2015, PLoS One 10(9):e0137455 and Olowu et al., 2010, Sensors 10(11):9872-90). These detection formats and instruments cannot be used with multi-well plates, which precludes their use in HTS laboratories. In addition, many aptamer-based assays have been developed using a solid phase format; i.e., they are not homogenous, and these assays are not well suited for high throughput screening (HTS) detection because they require wash steps that complicate automated workflow (Famulok & Mayer, 2014, Chem. Biol. 21(9):1055-8). Solid phase aptamer assays are most commonly formatted in a sandwich configuration, analogous to an antibody-based ELISA (Ochsner et al., 2014, Biotechniques 56(3):125-8, 130, 132-3). An immobilized aptamer is used to capture the analyte, several wash steps are used to remove non-specific molecules, and a second aptamer is then added which is attached to the signaling component either directly (e.g., a fluor) or via an affinity tag; e.g. streptavidin-biotin. These assays generally require as many as 15-20 wash steps, which greatly complicates their use in automated workflows, especially with high density plates such as 1536 well plates, making them impractical for HTS. Moreover, they require two aptamers that bind the analyte at separate epitopes. Unfortunately, many biological molecules of interest such as steroids and nucleotides are too small to accommodate binding to two aptamers simultaneously.

An alternative approach is the use of split aptamers (Chen et al., 2010, Biosens. Bioelectron. 25(5):996-1000). In this approach, an aptamer is split into two pieces, which re-associate in the presence of a target ligand. This re-association event provides an opportunity to engineer proximity based signaling mechanisms into aptamer sensors. This is especially advantageous for molecules that are too small for simultaneous binding of two aptamers, as it allows development of proximity-based sensors using a single aptamer. The split aptamer approach has been applied to a range of different aptamers and has been used to produce sensors for various molecules including small molecules such as cocaine, estradiol, adenosine and ATP, proteins; e.g., thrombin and whole cells (Liu et al., 2014, Sci. Rep. 4:7571; Park et al., 2015, Biosens. Bioelectron. 73:26-31; Qiang et al., 2014, Anal. Chim. Acta. 828:92-8; Yuan et al., Chem. Commun. (Camb.) 52(8):1590-3; Zhao et al., 2015, Anal. Chem. 87(15):7712-9; and Liu et al., 2014, ACS Appl. Mater. Interfaces 6(5):3406-12). In addition, a rational method for engineering a split site into existing aptamers was developed recently (Kent et al., 2013, Anal. Chem. 85 (29): 9916-23).

The use of a split aptamer approach offers distinct advantages for sensor development over structure-switching aptamers, as the only requirement is that the aptamer binds its target. However, aptamer based sensors developed thus far use solid phase detection and/or produce signals that are not compatible with HTS applications such as colorimetric, SPR, FI, or electrochemical signals (Liu et al., 2014, Sci. Rep. 4:7571; Liu et al., 2014, ACS Appl. Mater. Interfaces 6(5):3406-12; and Feng et al., 2014, Biosens. Bioelectron. 62:52-8). Therefore, there remains a need to develop aptamer based sensors conducive to performing HTS assays.

### SUMMARY OF THE INVENTION

It is against the above background that the present invention provides certain advantages and advancements over the prior art.

Although this invention disclosed herein is not limited to specific advantages or functionalities, the invention provides a sensor for measuring an analyte, comprising:

(a) a first fragment of a split aptamer and

(b) a second fragment of the split aptamer;

wherein the first fragment of the split aptamer comprises a first modification;

wherein the first fragment of the split aptamer and the second fragment of the split aptamer are associated in the presence of the analyte to form a trimeric complex with the analyte.

In one aspect of the sensors disclosed herein, the first fragment of the split aptamer and the second fragment of the split aptamer are DNA and/or RNA molecules.

In one aspect of the sensors disclosed herein, the DNA and/or RNA molecules comprise modified nucleotides.

In one aspect of the sensors disclosed herein, the first modification is a fluor modification.

In one aspect of the sensors disclosed herein, the fluor modification is a fluorescein, rhodamine, texas red, an alexa fluor, a cyanine dye, or an atto dye modification.

In one aspect of the sensors disclosed herein, the fluor modification is attached at a terminus of the split aptamer or internally in the split aptamer.

In one aspect of the sensors disclosed herein, the first modification is a streptavidin modification.

In one aspect of the sensors disclosed herein, a measured fluorescence polarization (FP) induced by the trimeric complex is larger than a measured FP induced by the first fragment of the split aptamer and the second fragment of the split aptamer prior to assembly of the trimeric complex.

In one aspect of the sensors disclosed herein, the second fragment of the split aptamer further comprises a second modification.

In one aspect of the sensors disclosed herein, the second modification is a luminescent lanthanide modification.

In one aspect of the sensors disclosed herein, the luminescent lanthanide is terbium or europium.

In one aspect of the sensors disclosed herein, the second modification is an upconversion nanoparticle.

In one aspect of the sensors disclosed herein, the trimeric complex produces a time-resolved fluorescence energy transfer (TR-FRET) signal.

The invention also provides a sensor for measuring an analyte, comprising:

(a) a first fragment of a split aptamer and

(b) a second fragment of a split aptamer;

wherein the first fragment of the split aptamer is conjugated to a first fragment of a reporter enzyme polypeptide;

wherein the second fragment of the split aptamer is conjugated to a second fragment of a reporter enzyme polypeptide;

wherein the first fragment of the split aptamer and the second fragment of the split aptamer are associated in the presence of the analyte to form a trimeric complex with the analyte.

In one aspect of the sensors disclosed herein, the first fragment of the split aptamer and the second fragment of the split aptamer are DNA and/or RNA molecules.

In one aspect of the sensors disclosed herein, the DNA and/or RNA molecules comprise modified nucleotides.

In one aspect of the sensors disclosed herein, the first fragment of the reporter enzyme polypeptide and the second fragment of the reporter enzyme polypeptide are complementary fragments of a split reporter enzyme.

In one aspect of the sensors disclosed herein, the first fragment of the reporter enzyme polypeptide and the second fragment of the reporter enzyme polypeptide assemble into an intact reporter enzyme in the presence of the analyte.

In one aspect of the sensors disclosed herein, the intact reporter enzyme is a luciferase protein.

In one aspect of the sensors disclosed herein, the first fragment of the reporter enzyme polypeptide has at least 90% identity to the amino acid sequence set forth in SEQ ID NO:15 and the second fragment of the reporter enzyme polypeptide has at least 90% identity to the amino acid sequence set forth in SEQ ID NO:16.

In one aspect of the sensors disclosed herein, the luciferase protein produces a luminescent signal upon conversion of a luciferin substrate.

In one aspect of the sensors disclosed herein, the analyte is an amino acid, an amino acid-related molecule, a peptide, a protein, a steroid, a lipid, a sugar, a carbohydrate, a drug molecule, a drug metabolite, a coenzyme, a nucleotide, a nucleotide-related molecule, a pyridine nucleotide, a cyclic nucleotide, or a cyclic dinucleotide.

In one aspect of the sensors disclosed herein, the analyte is S-adenosylhomocysteine (SAH).

In one aspect of the sensors disclosed herein, the analyte is a protein having a post-translational modification (PTM).

In one aspect of the sensors disclosed herein, the analyte is an acetylated and/or methylated histone.

The invention also provides a method for detecting an analyte, comprising:

(a) contacting the sensors disclosed herein with a sample;

wherein the first fragment of the split aptamer and the second fragment of the split aptamer assemble in the presence of the analyte to form a trimeric complex with the analyte; and

(b) measuring a signal generated upon assembly of the trimeric complex.

In one aspect of the methods disclosed herein, the signal generated is measured by FP, TR-FRET, and/or luminescence.

In one aspect of the methods disclosed herein, the analyte is detected in a high-throughput screen (HTS).

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1A is a schematic depicting a split aptamer FP assay, wherein binding of the ligand causes assembly of a trimeric complex resulting in increased polarization because of the larger size of the trimeric complex. See Examples 2 and 3. FIG. 1B is a schematic depicting a split aptamer TR-FRET assay, wherein binding of the ligand causes assembly of a trimeric complex resulting in a positive TR-FRET signal because of the increased proximity of the lanthanide donor to the organic fluor acceptor. See Example 4. FIG. 1C is a schematic depicting a split aptamer EFC assay, wherein binding of the ligand induces a split aptamer to assemble into a trimeric complex, resulting association of the two halves of a signaling enzyme (E1 and E2). The intact enzyme converts its substrate to a detectable product. See Example 7.

FIG. 2A shows a predicted structure of R. ferrireducens metH riboswitch (SEQ ID NO:1) in the absence of SAH showing sequestration of the Shine Dalgarno (SD) sequence. FIG. 2B shows a conformational switch mechanism based on the crystal structure of SAH-bound riboswitch from R. solanacearum, showing formation of a more ordered structure upon SAH binding accompanied by exposure of the SD sequence.

FIG. 3A shows FP signaling with intact riboswitch, where SAH binding causes a conformational shift that exposes a single strand region, allowing hybridization of a fluorescently labeled oligo and increasing polarization. FIG. 3B shows dose responses for SAH, SAM, and ATP, where K_{d} values increased over time, indicating instability in riboswitch structure. See Example 1.

FIGS. 4A-4D show a split aptamer SAH FP Assay. FIG. 4A shows that SAH dependent assembly of split aptamer (Dar1 P1₅₉/P2₁₈) causes increased polarization of a fluor attached to the P2₁₈ element. FIG. 4B shows an SAH titration, including concentration dependence and stability of signal. FIG. 4C shows a comparison of response to SAH, SAM and ATP. FIG. 4D shows a standard curve mimicking enzymatic conversion of 200 nM SAM to SAH. FIG. 4E shows an SAH/SAM titration, showing concentration dependence of signal and comparison of response to SAH and SAM. FIG. 4F shows standard curve mimicking enzymatic conversion of 200 nM SAM to SAH in the presence of various HMT acceptor substrates. See Example 2.

FIGS. 5A-5F show detection of histone methyltransferases (HMTs) with a split aptamer FP Assay. FIG. 5A shows detection of HMT PRMT3 with 500 nM SAM. FIG. 5B shows linearity of response to enzyme concentration; polarization data from FIG. 5A was converted to SAH formation. FIG. 5C shows a time course of SAH formation at 500 nM SAM. FIG. 5D shows detection of HMT PRMT3 with 100 nM SAM. FIG. 5E shows SAH formation by PRMT3 at 100 nM SAM; polarization data from FIG. 5D was converted to SAH. FIG. 5F shows SAM Kₘ determination for PRMT1. See Example 3.

FIGS. 6A-6H show a split aptamer SAH TR-FRET assay. FIG. 6A shows SAH-driven assembly of split aptamer allows FRET between Tb donor and organic dye acceptor. FIGS. 5B and 5C show SAH dose response curves for assays using Eu/Alexa633 and Tb/Alexa633 as donor/acceptor, respectively, showing concentration dependence and stability of signal over time. FIG. 6D shows a standard curve mimicking enzymatic conversion of 200 nM SAM to SAH with Tb/Dylight 650 as donor/acceptor. FIG. 6E shows detection of PRMT4 with full length histone acceptor, 200 nM SAM. FIG. 6F shows a time course for PRMT4 reaction at 6 ng/μL; data from FIG. 6E was converted to SAH formation. FIG. 6G shows detection of NSD2 with nucleosome acceptor, 2 μM SAM, 2 h incubation. FIG. 6H shows detection of DNMT1 with poly-dl-dC acceptor. See Example 4.

FIG. 7 depicts a schematic of the split aptamer luminescence assay. Specific epigenetic marks induce a split aptamer to assemble into a trimeric complex, resulting in activation of split luciferase (Luc) and production of an amplified luminescence signal.

FIG. 8A shows the predicted folded structure of an H4K16Ac aptamer (SEQ ID NO:2). FIG. 8B shows the predicted folded structure of an H3R8Me2sym aptamer (SEQ ID NO:3). The arrows in FIGS. 8A and 8B show potential sites for splitting.

Skilled artisans will appreciate that elements in the figures are illustrated for simplicity and clarity and have not necessarily been drawn to scale. For example, the dimensions of some of the elements in the figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “polynucleotide,” “nucleotide,” “oligonucleotide,” and “nucleic acid” can be used interchangeably to refer to nucleic acid comprising DNA, RNA, derivatives thereof, or combinations thereof.

As used herein, the term “and/or” is utilized to describe multiple components in combination or exclusive of one another. For example, “x, y, and/or z” can refer to “x” alone, “y” alone, “z” alone, “x, y, and z,” “(x and y) or z,” “x and (y or z),” or “x or y or z.”

As used herein, the term “aptamer” can be used to refer to a molecule that can bind to a specific target with high specificity and affinity. The aptamer can be an oligonucleotide, such as DNA or RNA, or a peptide. In particular, the aptamer can be a single-stranded oligonucleotide, such as single-stranded DNA.

Aptamers are nucleic acid affinity reagents that have been developed for detection of diverse ligands ranging in size from small molecules (cocaine, thalidomide, ATP, dopamine) to proteins (VEGF, EGFR) to cells (McKeague & Derosa, 2012, J Nucleic Acids 2012:748913; Yuce et al., 2015, Analyst 140(16):5379-99). As used herein, the term “split aptamer” can be used to refer to an aptamer that is composed of two or more fragments. For example, a split aptamer can be composed of two fragments, i.e. P1 and P2. Split aptamers retain specificity for their targets (Sharma et al., 2011, J Am. Chem. Soc. 133(32):12426-9) and have been shown to recognize a variety of molecules such as thrombin (Chen et al., 2010, Biosensors and Bioelectronics 25(5):996-1000), adenosine (Yang et al., 2011, Analytical Methods 3(1):59-61 and Wang et al., 2011, Sensors and Actuators B: Chemical 156(2):893-8), ATP (Liu et al., 2010, Chemistry-A European Journal 16(45):13356-9 and He et al., 2013, Talanta 111:105-110), and cocaine (Sharma et al., 2012, Analytical Chemistry 84(14):6104-9) and may also improve the detection sensitivity as compared to intact aptamers (Liu et al., 2014, Sci. Rep. 4:7571). Moreover, the length of an intact aptamer is not thought to be limiting factor, since split aptamers generated from relatively short 15-mer thrombin or 27-mer ATP aptamer are capable of self-assembly (Chen & Zeng, 2013, Biosensors and Bioelectronics 42:93-99). Typically, a full-length aptamer is split into two parts in a way such that the target molecule bound in the pocket forms a bridge between the two split fragments.

The split aptamers utilized herein can be conjugated to a dye, such as an organic donor fluor or an organic acceptor fluor, a luminescent lanthanide, a fluorescent or luminescent nanoparticle, an affinity tag such as biotin, or a polypeptide. The fluor can be, for example but not limited to, fluorescein, rhodamine, Texas Red, Alexa Fluors such as AlexaFluor 633 and AlexaFluor 647, Cyanine dyes such as Cy3 and Cy5, or Atto dyes such as Atto 594 and Atto 633. The nanoparticle can be an upconversion nanoparticle (see Wang et al., 2016, Analyst 141:3601-20). The polypeptide can be a reporter enzyme, such as a luciferase polypeptide. A luminescent lanthanide can be attached to a split aptamer by interactions not limited to a streptavidin-biotin interaction, a His-tag-metal interaction, or by covalent attachment.

As used herein, the term “riboswitch” can be used to refer to a structured noncoding RNA molecule capable of binding to an analyte and/or regulating gene expression. As used herein, riboswitches are microbial metabolite sensing RNA aptamers.

As used herein, the terms “homogenous assay,” “homogenous format,” and “homogenous detection” can be used to refer to detection of an analyte by a simple mix and read procedure. A homogenous assay does not require steps such as sample washing or sample separation steps. Examples of homogenous assays include TR-FRET, FP, FI, and luminescence-based assays.

As described herein, aptamers offer significant advantages over antibodies as affinity reagents for biomolecular detection. First, aptamers are typically generated using an in vitro selection process called SELEX (Systematic Evolution of Ligands by EXponential enrichment, which is a key advantage over the lengthy in vivo methods used to generate antibodies (McKeague & Derosa, 2012, J Nucleic Acids 2012:748913). SELEX can be performed in a matter of days, and unlike in vivo antibody production, it does not require that the target molecule be conjugated to a carrier protein. The affinity and specificity of aptamers can be further enhanced following the initial selection using rapid in vitro methods such as site directed mutagenesis and directed evolution, an approach that was recently used to increase the selectivity of a histone H4 aptamer more than 20-fold (Yu et al., 2011, Chembiochem. 12(17):2659-66). Aptamers are also less expensive to produce and have lower batch-to-batch variation compared to antibodies. In addition, they are much easier to engineer and modify in specific ways than antibodies, such as incorporation of fluorophores at specific sites, because most desired changes can be introduced during solid state synthesis (Juskowiak, 2011, Anal. Bioanal. Chem. 399(9):3157-76). In contrast, specific labeling of antibodies often requires insertion of non-native amino acids, which is extremely time consuming and requires specialized expertise, and the results are difficult to predict (Sochaj et al., 2015, Biotechnol. Adv. 33(6 Pt 1):775-84).

To be practically useful in biomedical research applications, an aptamer based assay must be useful in high throughput applications such as screening chemical libraries for potential drug molecules or testing large numbers of biological samples for the presence of disease biomarkers (Kong et al., 2012, J. Lab. Autom. 17(3):169-85; Nicolaides et al., 2014, Front. Oncol. 4:141; and Hughes et al., 2011, Br. J. Pharmacology 162(6):1239-49). Utility for HTS applications imposes strict requirements on aptamer based assays, most notably that they are configured in a homogenous or “mix-and-read” format and that they produce a signal that provides sensitive detection with minimal interference using instruments commonly found in HTS laboratories (Hughes et al., 2011, Br. J. Pharmacology 162(6):1239-49 and Jones et al., 2004, Assay Guidance Manual, Ed. Sittampalam et al.).

One of the main advantages of aptamers over antibodies for detection is that the signaling component can be integrated into the aptamer itself to produce a sensor. The advantage of sensors is that they provide direct detection of analytes without the use of additional detection reagents. This inherent simplicity is advantageous both from an assay development standpoint and for practical use. Sensors do not require the development of additional reagents, such as a second aptamer for solid phase assay or a tracer for competitive assays. In addition, aptamer-based sensors are generally formatted for homogenous detection, which makes them well suited for HTS. This is especially advantageous for molecules that are too small to accommodate binding of two antibodies for a sandwich assay format. Detection of these small molecules with antibodies requires competitive assays, such as competitive ELISAs, or radioimmunoassays (RIAs). The use of RIAs is highly undesirable due to radiation hazards and the associated regulatory and disposal costs. Competitive assays are undesirable because they generally produce a negative signal, and thus are not as sensitive and have a limited dynamic range.

An exemplary signaling mechanism used for aptamer-based sensors is a change in the properties of an attached fluor upon analyte binding. Ligand binding often induces structural shifts in the aptamer which can change the microenvironment of attached fluors resulting in quenching, enhanced emission, or changes in polarization. For example, for aptamers that bind proteins, a fluor attached to the aptamer usually undergoes an increase in polarization upon formation of the protein-aptamer complex because of the slower rotational mobility of the complex relative to the free aptamer. Alternatively, a fluor can be attached to a complementary oligonucleotide that undergoes displacement or, less commonly, annealing, due to ligand-induced structural shifts in the aptamer. A common configuration for such oligo-displacement assays is to attach a quencher to one element (i.e., the aptamer or the complementary oligo) and a fluor to the other, such that ligand induced dissociation of the oligo results in enhanced fluorescence. For reasons that are not fully understood, it is sometimes not possible to produce robust aptamer based sensors using a structure switching approach. Though efforts in this direction are ongoing, the difficulty in developing structure switching aptamers remains a significant hurdle in development of aptamer based sensors.

As described herein, sensors were developed using a split aptamer configuration to produce positive TR-FRET, FP, and luminescent signals upon binding of a target ligand. Upon splitting of an aptamer by breaking a covalent phosphodiester bond, the two fragments of the aptamer only associate if enough complementary bases in the two fragments allow for annealing. Additionally, the split aptamer fragments must be present at sufficiently high concentrations to drive the equilibrium toward annealing. From a thermodynamics standpoint, the energy of the trimeric complex is lower than the energy of the free components. The lower energy comes from the bonds between the ligand and the aptamer, which can be, for example, ionic or Van der Waals.

These sensors can be used for highly sensitive detection of biomolecules, including small molecules and proteins, in a homogenous format. In some embodiments, the split aptamer sensors produce signals that can be detected with commonly used multimode plate readers. Surprisingly, splitting of an aptamer into two fragments, such that ligand binding induces assembly of a trimeric complex, improves the sensitivity, selectivity, and stability of signaling, as compared to a structure switching sensor.

In some embodiments, a split aptamer assay is performed with FP readout. In the FP based assay, one of the two aptamer fragments, called P1 and P2, is labelled with a fluor. In the presence of the target ligand, P1 and P2 form a trimeric complex with the ligand. This results in a decrease in the rotational mobility of the attached fluor, causing its polarization to be increased. In some embodiments, changes in the microenvironment of the fluor in the trimeric complex increase the magnitude of the polarization signal. See Examples 2 and 3.

In some embodiments, a split aptamer assay is performed with a TR-FRET readout. One of the two aptamer fragments can be conjugated to a lanthanide chelate, and the other can be conjugated to an organic fluor acceptor. In the absence of the target ligand, P1 and P2 remain largely unassociated, and therefore energy transfer from the lanthanide to the organic fluor is minimal. In the presence of the target ligand, P1 and P2 form a trimeric complex with the ligand, which brings the lanthanide and organic fluor in close enough proximity to allow substantial transfer of energy from the lanthanide to the fluor, resulting in a strong TR-FRET signal. See Example 4.

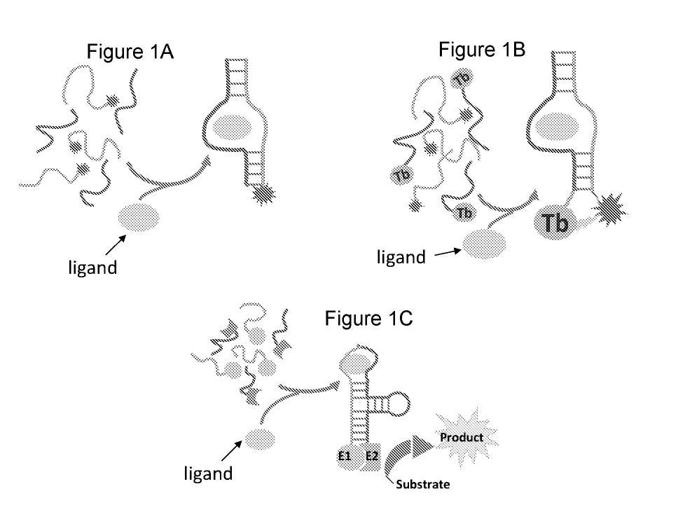

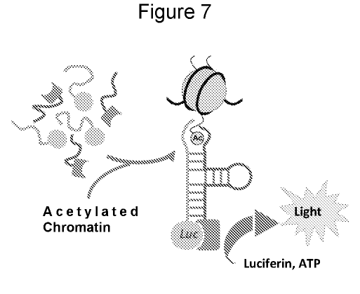

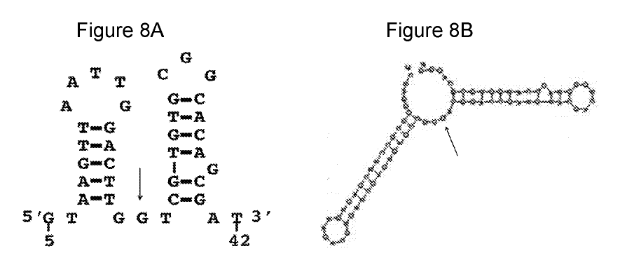

In some embodiments, a split aptamer assay is performed with a luminescence readout. In some embodiments, split aptamers are combined with enzyme fragment complementation (EFC) using a split luciferase to provide a luminescence signal (FIGS. 1C and 7), which is suitable for use with cell lysates and tissue samples. Each of the aptamer fragments is conjugated to a fragment of a luminescence-producing enzyme, such as a luciferase enzyme. In the absence of the target ligand, P1, P2 and the attached enzyme fragments remain largely unassociated, therefore; luminescence is minimized. In the presence of the target ligand, P1 and P2 form a trimeric complex with the ligand, which brings the two luminescent enzyme fragments in close enough proximity to associate and restore the enzyme's catalytic activity in the presence of the appropriate substrates and cofactors. See Example 7.

EFC has been extensively employed to study protein-protein interaction in cells and in vitro by recombinantly fusing the two proteins of interest to the two split enzyme fragments (Michnick et al., 2007, Nature Rev. Drug. Disc. 6(7):569-82; Paulmurugan & Gambhir, 2003, Analytical Chemistry 75(7):1584-9; and Luker et al., 2004, Proc. Natl. Acad. Sci. USA 101(33):12288-93). The bioluminescent enzyme luciferase can be fragmented into two parts, NLuc and CLuc representing the N-terminal and C-terminal of the enzyme, respectively (Paulmurugan & Gambhir, 2003, Analytical Chemistry 75(7):1584-9; Luker et al., 2004, Proc. Natl. Acad. Sci. USA 101(33):12288-93; and Porter et al., 2008, J Am. Chem. Soc. 130(20):6488-97). Individually, the two enzyme fragments are inactive; however, upon target recognition they are brought into proximity and the enzyme activity is restored (FIG. 1C). Generating EFC reagents for protein-protein interaction assays requires re-optimization of the recombinant expression and purification conditions for each fusion construct. However, the approach described herein relies on expressing the split-luciferase enzyme fragments alone, and in a second step, synthetically conjugating split aptamer to them. Proximity-based restoration of luciferase activity (EFC coupled to protein-protein interaction assays) has been demonstrated for a number of proteins such as VEGF, GTPase-activating proteins (GAP), and guanine-nucleotide-exchange factors (GEF), and nuclear-factor-e2-related transcription factor 2 activators (Nrf2) (Stains et al., 2010, ACS Chem. Bio. 5(10):943-52; Erik & Michael, 2012, Biochemical Journal 441(3):869-79; and Xie et al., 2012, Assay Drug Dev. Technol. 10(6):514-24). Moreover, use of synthetic nucleic acid-Luc constructs, as opposed to vector expressed fusion proteins, can enable rapid development of new assays for additional targets.

In some embodiments, split aptamer technology as described herein can be used for detection of biomolecules in live cell cultures. For example, cells grown in multi-well plates are commonly used to screen potential drug molecules for their effects on signaling pathways involved in disease pathogenesis. A common endpoint for these cellular assays is detection of soluble factors such as inflammatory cytokines, growth factors or steroid hormones. The FP, TR-FRET and luminescent aptamer sensors could be added directly to the wells for in situ detection of soluble signaling molecules. This provides a significant advantage over the alternative approach of transferring aliquots of media from the wells to separate plates for ELISA-based assays. The initial liquid transfer step for ELISA assays introduces a source of error, and the subsequent wash steps make the assay very cumbersome for an automated HTS platform. In contrast, the addition of aptamer sensors directly to the cell culture wells requires only one liquid handling step, making it easy to automate and allowing accurate measurement of analyte levels in situ.

In some embodiments, split aptamer technology as described herein can be used for detection of biomolecules that are biomarkers for disease prognosis or for predicting therapeutic response to specific drugs (i.e., companion diagnostics). Such assays have become a critical part of new drug development as they allow selection of patients for clinical trials that are likely to respond to the drug being tested. In addition, biomarker assays can be used to select the best drug or combination of drugs for treatment of patients. Though antibody-based assays can be used for many protein biomarkers, detection of small molecule biomarkers such as amino acids, sugars, or nucleotides is complicated by the lack of sensitive, homogenous assay methods. The use of antibodies for small molecule detection requires competitive and/or radioactive assays which are highly undesirable for automated HTS applications. The FP, TR-FRET, and luminescent aptamer sensors could be added directly to biological fluids such as serum, urine, or saliva for detection of small molecule biomarkers using multimode plate readers or similar instruments commonly found in clinical research and diagnostic laboratories.

In some embodiments, split aptamer technology as described herein can be used for measuring enzyme activity by detection of reaction products. Enzymes such as kinases and methyltransferases that have been shown to be involved in disease pathogenesis are often screened in HTS laboratories to identify inhibitors or activators that can potentially be developed into drug molecules. In this aspect, the FP, TR-FRET, and luminescent aptamer sensors that recognize the product of an enzyme reaction can be added directly to wells of plates, and the signal could be read on the multimode readers commonly used in HTS laboratories. Use of the aptamer based sensors can be useful in cases where the enzyme product being detected is a small molecule such as a nucleotide, an amino acid, or a steroid.

Use of the split aptamer assays described herein is preferable over use of competitive displacement assays for the detection of small analytes. In a competitive displacement assay, a detection tag such as a fluor or reporter enzyme is attached to the analyte to produce a tracer. Displacement of the tracer from the aptamer by an analyte causes a change in its signal. For example, if the aptamer is immobilized, the detection tag is released into the soluble fraction, which can be sampled separately from the bound aptamer. Alternatively, if a fluorescent tag is used, its optical properties (i.e., brightness or polarization) may change upon displacement, allowing a homogenous assay format. These effects can be enhanced by using quencher-acceptor pairs on the tracer and the aptamer. Though such competitive assays can be formatted for homogenous detection, tracer development is often problematic because attachment of detection tags to an analyte usually decreases its affinity to the aptamer or disrupts binding completely. This can greatly complicate or prevent development of competitive displacement assays, especially as the size of an analyte decreases.

The split aptamer assays described herein can be used to detect biomolecules, for example, but not limited to amino acids and amino acid related molecules such as dopamine and thyroxine, peptides and proteins, steroids, lipids, sugars and carbohydrates, drug molecules and their metabolites, coenzymes such as acetyl-coenzyme A and cobalamin, nucleotides and nucleotide-related molecules such as nucleotide nucleotide-diphospho-sugars, pyridine nucleotides (NAD and NADH), cyclic nucleotides and cyclic dinucleotides.

In one example, the split aptamer assays described herein can be used to detect protein and DNA modifications, such as histone methylation or DNA methylation. Histone methyltransferases are rapidly emerging as promising therapeutic targets for diverse diseases, especially cancer. Histone and DNA modifications play critical roles in normal development as well as susceptibility to diverse diseases including diabetes, cardiovascular diseases, cancers, and inflammatory diseases (Day & Sweatt, 2012, Neuropsychopharmacology 37(1):247-60; Reddy & Natarajan, 2011, Cardiovasc. Res. 90(3):421-9; Werda et al., 2010, J. Cell. Mol. Med. 14(6A):1225-40; and Copeland et al., 2009, Nat. Rev. Drug Discovery 8(9):724-32). Drug discovery efforts targeting methyltransferases are intense and growing rapidly, partially because the clinical success with HDAC inhibitors provides validation for epigenetics targets in general (Copeland et al., 2009, Nat. Rev. Drug Discovery 8(9):724-32)). Two DNA methyltransferases (DNMT) inhibitors have been approved as drugs and additional compounds are in trials for various cancers, however current drug discovery efforts are focused mostly on histone methyltransferases (HMTs) (Bouchie, 2012, Epigenetics Land Grab, Biocentury).

In some embodiments, the split aptamer assays described herein detect S-Adenosyl-L-homocysteine (SAH), the product of all S-Adenosyl-L-methionine (SAM)-dependent methyltransferase reactions. In some embodiments, the split aptamer assays described herein have a higher sensitivity for SAH than currently available methods. Development of assays to measure methyltransferase assays is more difficult than development of kinase assays. Methyltransferases are generally very slow enzymes, with turnovers in the range of less than 1 min⁻¹ in many cases, and they tend to have low Kₘ values for SAM, many in the sub-micromolar range (Janzen et al., 2010, Drug Discov. Today Technol. 7(1):e59-65). These properties impose very high sensitivity requirements on assay methods, requiring detection of low nanomolar levels of product under typical screening conditions (initial velocity conditions using Kₘ concentrations of SAM.) For instance, a number of HMTs have SAM Kₘs of 80-100 nM, which requires detection of 5-20 nM SAH for initial velocity measurements. Moreover, high enzyme production costs are another impetus for more sensitive detection methods, as many HMTs function as complexes, with three or four proteins required for full activity.

Detection of SAH is advantageous over detection of methylated products for a number of reasons. Whereas kinases catalyze mono-phosphorylation, HMTs can add up to two methyl groups at arginines and up to three at lysines, resulting in a total of six possible methylation states. The diversity of methylated reaction products combined with variability in surrounding amino acids complicates immunochemical assay methods, as a single antibody generally does not recognize all of the products formed by a single HMT (Janzen et al., 2010, Drug Discov. Today Technol. 7(1):e59-65). Moreover, the development of specific antibodies is not keeping pace with the discovery of new methylation sites, and assay development is being prevented in some cases. Methyl binding domains typically have very low affinity, and thus do not afford sensitive detection. However, although SAH detection provides a simpler, universal HMT assay method, significant technical gaps have thus far prevented development robust, highly sensitive SAH detection assays. Though antibodies have been developed to discriminate between SAH and SAM (Graves et al., 2007, Anal. Biochem. 373:296-306), they lack the affinity required for a highly sensitive HMT assay. Recently, an SAH immunodetection assay with a TR-FRET readout was introduced by CisBio with a stated lower limit of 400 nM SAM (CisBio BioAssays, Codolet, France), which is several-fold higher than required for HMTs with low Kₘs (e.g., 80-100 nM). The primary approach used in commercial assays is enzymatic conversion of SAH to a molecule that can be detected directly; i.e., enzyme coupled assays. There are several versions of the coupled enzyme assays, which are less sensitive and/or more prone to interference than the split aptamer assays described herein. See, e.g., Collazo et al., 2005, Anal Biochem. 342(1):86-92; Wang et al., 2005, Biochem. Biophys. Res. Commun. 331(1):351-6; Dorgan et al., 2006, Anal. Biochem. 350(2):249-55; Hendricks et al., 2004, Anal. Biochem. 326(1):100-5; and Ibanez et al., 2010, Anal. Biochem. 401(2):203-10.

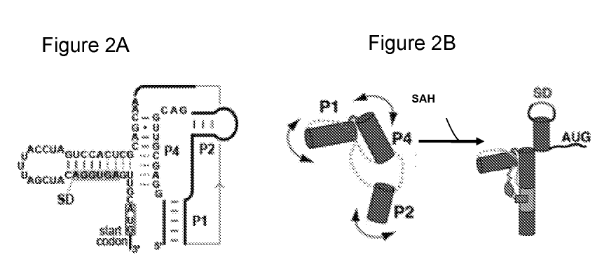

To overcome the technical gap in HMT HTS assays, riboswitches were used to develop FP and TR-FRET-based SAH sensors. Highly specific SAH riboswitches (FIG. 2) have been found located upstream of operons for one of three different SAH recycling genes, which they regulate by interacting with translational or transcriptional control elements (Wang et al., 2008, Mol. Cell. 29(6):691-702). A representative SAH riboswitch from D. aromatica was characterized using equilibrium binding analysis and found to have a K_{d} of 20 nM for SAH and an affinity for SAM that was at least 1000-fold lower (Wang et al., 2008, Mol. Cell. 29(6):691-702), and another from R. soanacearum was shown to have a K_{d} for SAH of 30 nM using isothermal titration calorimetry (Edwards et al., 2010, RNA 16(11):2144-55). Binding studies with SAH analogs (Wang et al., 2008, Mol. Cell. 29(6):691-702) and subsequent structural studies (Edwards et al., 2010, RNA 16(11):2144-55) have shown that virtually every functional group in the SAH molecule interacts with the SAH riboswitch, which is consistent with the binding characteristics of other metabolite riboswitches (Montange et al., 2008, Annu. Rev. Biophys. 37:117-33). These stringent binding requirements are ideal for a methyltransferase HTS assay as they can enable detection of very low amounts of SAH in the presence of excess SAM with very little chance of interference from SAH-competitive inhibitors.

As described in Examples 2-4, SAH binding was transduced into stable (>12 h) FP and TR-FRET signals using a split aptamer format. Selectivity for SAH vs. SAM of at least 200-fold was achieved, which is sufficient for measuring HMT initial velocity. Detection of low nM SAH concentrations enabled HMT activity measurements using 100 nM SAM. Detection of enzyme activity with peptide, histone, nucleosome, and DNA substrates and determination of kinetic parameters (e.g., Kₘ, Vₘ) validated the split aptamer assays for detection of diverse HMTs. The maintenance of a strong signal for over 12 h and the robust enzyme detection results indicated that the split aptamer is sufficiently stable for use in HTS assays.

In some embodiments, a split aptamer assay can be used to detect post-translational modifications (PTMs). Epigenetic regulation has been implicated in diverse diseases including cancer, diabetes and inflammation, and specific detection of histone PTMs, especially methylation and acetylation, is fundamental to basic research and drug discovery in this area. The enzymes that catalyze PTM reactions, predominantly kinases and more recently histone modifying enzymes, are the targets of 30-40% of current pharma/biotech drug discovery efforts. Changes in a specific PTM is the most frequently used biomarker to confirm target engagement in translational studies of PTM enzyme inhibitors, and increasingly as diagnostic biomarkers for disease (Sandoval et al., 2013, Expert Rev. Mol. Diagn. 13(5):457-71 and Pierobon et al., 2015, Oncogene 34(7):805-14). However, the fundamental analytic requirement for understanding how PTMs affect cell function and disease—unambiguous detection of specific PTMs in complex mixtures—remains a significant technical challenge, especially in a format amenable to automated HTS.

Immunodetection methods, though widely used, are not keeping pace with the growing demand for epigenetic biomarker assays. In many cases, antibodies lack the specificity required to discriminate between subtle and complex histone PTMs, and the assay methods are cumbersome and expensive. In addition to the recognition challenges, immunodetection of PTMs generally requires separation steps such as chromatography, gel electrophoresis, or solid phase assays with wash steps, e.g., ELISA, which are cumbersome to incorporate into automated HTS workflows. In proximity based methods, such as MesoScale (Gowan et al., 207, Assay Drug Dev. Technol. 5(3):391-401), the wash steps are eliminated, however they require two antibodies for each target and specialized plates, which makes the technology very expensive. Homogenous fluorescent detection methods that rely on FRET between two different antibodies to the target protein are used for phosphoprotein detection, e.g., HTRF (Ayoub et al., 2014, Front. Endocrinol. (Lausanne) 5: 94), but this is an expensive, complicated approach, and it has yet been applied to epigenetic PTMs. Methyl binding domains; e.g. bromodomains, have shown promise as specific detection reagents, but they typically have low affinity, and thus do not allow highly sensitive detection (Kungulovski et al., 2014, Genome Res. 24(11):1842-53). Thus, in some embodiments, the affinity and specificity of nucleic acid aptamers can be used to develop a platform for homogenous detection of epigenetic PTMs in cell and tissue samples to overcome the challenges associated with currently available methods.

In some embodiments, the split aptamers described herein allow for development of an economical, easily automatable assay platform for unambiguous identification of epigenetic marks in cells and tissues, which can fill a significant unmet need in epigenetic drug discovery and accelerate efforts to target chromatin modifying enzymes for cancer and other diseases. In some embodiments, the ability to generate new, highly selective aptamers in vitro in a matter of days combined with the low cost and high reproducibility of oligonucleotide synthesis methods can allow for detection of diverse epigenetic marks, such as methylation, acetylation phosphorylation, glycosylation, ubiquitination, and sumoylation.

In some embodiments, an aptamer specific for epigenetic PTMs is used. In one example, the aptamer can be a 50 base RNA aptamer developed for dimethyl Arg in Histone H3 (H3R8Me2), which generated through ten cycles of SELEX using a 14 amino acid peptide comprising the target PTM. The H3R8Me2 aptamer can have a high affinity of 12 nM for the target modified peptide but only moderate selectivity (3.5-fold and 8.1-fold, respectively) for the unmodified peptide and a similar histone PTM (H3K9Me₂) (Hyun et al., 2011, Nucleic Acid Ther. 21(3):157-63). In another example, the aptamer can be a 48 base DNA aptamer for histone H4 acetylated at lysine 16 (H4K16Ac), which was generated with four rounds of SELEX that included a negative selection against an unmodified H4K16 peptide. The H4K16Ac aptamer can bind to its target PTM with a K_{d} of 21 nM and can have more than 2,000-fold selectivity versus a very similar Histone H4 acetylation (at K8) or an unmodified Histone H4 sequence (Williams et al., 2009, J Am. Chem. Soc. 131(18):6330-1). In some embodiments, due to their small size, aptamers such as the above-described aptamers demonstrate more efficient binding to a target PTM adjacent to other PTMs than antibodies do (Kungulovski et al., 2014, Genome Res. 24(11):1842-53). See Example 5.

In some embodiments, selection and optimization of split aptamer fragments for an assay with a luminescence readout is performed, which are important steps since the two fragments (P1 and P2) play a dual role in target recognition and in driving reassembly of split luciferase fragments. Specifically, a split aptamer pair can be developed that reassembles in the presence of a target PTM. See Example 6. In some embodiments, split aptamers are combined with EFC using a split luciferase to identify a PTM. See Example 7.

### EXAMPLES

The Examples that follow are illustrative of specific embodiments of the invention, and various uses thereof. They are set forth for explanatory purposes only, and are not to be taken as limiting the invention.

### Example 1: Demonstration of an SAH Dependent Switch with FP Readout

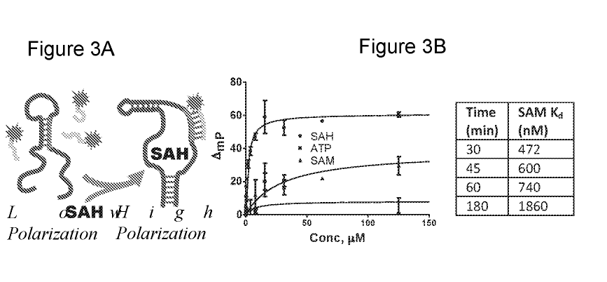

The native riboswitch signaling mechanism was first leveraged by transducing the conformational switch that occurs upon SAH binding into a fluorescent signal. The goal was to identify an oligonucleotide that would bind to the riboswitch specifically when SAH was present due to the localized conformational disruption it causes (FIG. 3A). Using two well characterized riboswitches, Ref-1 (SEQ ID NO:1) and Dar-1 (SEQ ID NO:4) (Wang et al., 2008, Mol. Cell. 29(6):691-702), an unbiased, empirical approach was taken by testing a panel of 8-10 bp oligos that covered the entire sequence of each riboswitch. The oligos were labeled with fluors at the 5′ end so that binding could be detected by the increase in fluorescence polarization. Surprisingly, only one of the 10-12 oligos tested (5′CY5-GAGCGCCGUU-3′; SEQ ID NO:5) for each riboswitch showed SAH dependent binding; it was homologous to a region of sequence identity and was functional with both riboswitches.

A robust, dose dependent FP shift under optimized conditions was observed, which was quite reproducible. However, the affinity of the riboswitch for SAH was much lower than the low nanomolar level expected, and it decreased further over time (see FIG. 3B and inset table for representative data with Dar-1). For instance, for the Dar-1 riboswitch, the initial EC₅₀ for SAH was 472 nM, and it increased more than 4-fold to 1.9 μM after 3 h. Moreover, the selectivity for SAH vs. SAM was much poorer than expected, less than 50-fold for both Dar-1 and Ref-1 rather than the 1000-fold-plus selectivity reported for the native riboswitch (Wang et al., 2008, Mol. Cell. 29(6):691-702). Despite significant effort to understand and eliminate the unstable nature of the SAH-riboswitch interaction, including confirming that both the riboswitch and the SAH were not degrading over time, a stable, high affinity SAH dependent signal was unable to be obtained. Thus, a split aptamer approach was next explored.

### Example 2: Development of a Novel FP-Based Split Aptamer Assay for High Affinity, Selective SAH Detection

The SAH riboswitches used were 60-70 bases, which are longer than the 20-40 base aptamers that have been used for other biosensors (Liu et al., 2014, Sci. Rep. 4:7571). There have been reports of low sensitivity when longer aptamers are used as biosensors (Liu et al., 2014, Sci. Rep. 4:7571 and Park et al., 2015, Biosens. Bioelectron. 73:26-31). Accordingly, a split aptamer approach was tested, which was recently used to increase the sensitivity of a long (76 base) DNA aptamer biosensor for estradiol (Liu et al., 2014, Sci. Rep. 4:7571). Notably, split aptamers have also been used to detect other small molecules similar to SAH, including adenosine and ATP (Park et al., 2015, Biosens. Bioelectron. 73:26-31).

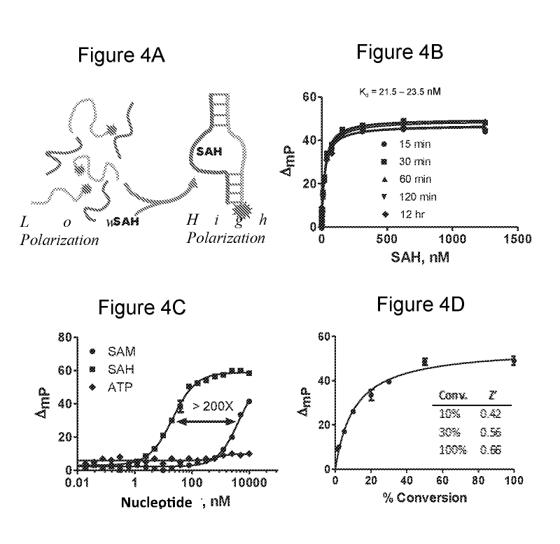

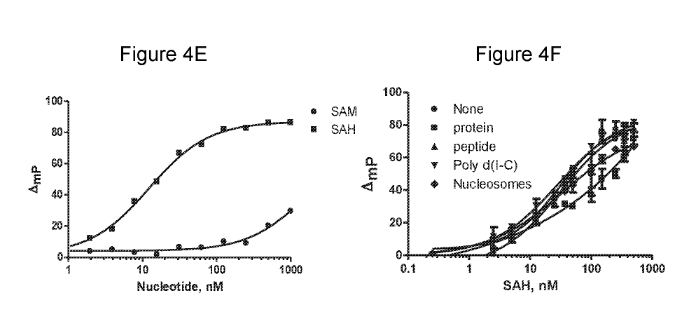

Based on the predicted folding of the Dar-1 sequence and imputed SAH binding interactions (Wang et al., 2008, Mol Cell. 29(6):691-702), two versions of a split Dar-1 riboswitch were tested, comprised of either a 59 or 52 base 5′ element (P1₅₉ of SEQ ID NO:6 or P1₅₂ of SEQ ID NO:7) and the remaining 18 base 3′ element (P2₁₈ of SEQ ID NO:8). Concentration dependent increases in polarization were observed with both split aptamers as SAH was added, indicating assembly of the two parts into a complex with the ligand (data for P1₅₂/P2₁₈ in FIG. 4B). The maximum FP shift was observed with equimolar amounts of the P1 and P2 elements in the 10-30 nM range, and the t_{1/2} to reach equilibrium was approximately 15 min at room temperature. The EC₅₀ values for SAH, calculated from the FP dose response curves in FIG. 4B, were 20-25 nM, which was in the expected range. For simplicity, EC₅₀ values were used as an approximation of ligand dissociation constants, or K_{d}. The SAH binding signal generated by the split aptamers was constant for at least 24 h at room temperature (FIG. 4B), indicating that the instability issues observed with the intact riboswitches in Example 1 had been eliminated. This is the first example of a split aptamer sensor with FP readout.

Selectivity vs. SAM is a critical parameter, as measurement of HMT enzyme activity requires detection of SAH in an excess of SAM. In this regard, ATP and SAM were much less effective at complexation with the split aptamers, with EC₅₀ values of more than 4,000 (FIG. 4C). Thus, the selectivity for SAH is at least 200-fold and likely higher; however, measurements are limited by contamination of SAM with SAH. The combination of sensitivity and selectivity is reflected in a standard curve mimicking an HMT enzyme reaction, in which 200 nM SAM was decreased as SAH was added proportionately (FIG. 4D). To quantitatively assess the robustness of the split aptamer FP assay, the standard curve was done with sixteen replicates to allow determination of Z′ values, a commonly used HTS assay statistic that measures that incorporates both dynamic range and data variability (Zhang et al., 1999, J Biomol. Screen. 4(2):67-73). A Z′ of greater than 0.5 is generally considered to indicate a robust, high quality assay. The Z′ at 10% conversion (i.e., 20 nM SAH/180 nM SAM) was 0.42, and at 30% conversion, which many investigators would consider acceptable for HTS. The Z′ measured in this Example was 0.56, indicative of a high quality assay.

### Example 3: Detection of HMT Enzyme Activity with the Split SAH Aptamer Biosensor

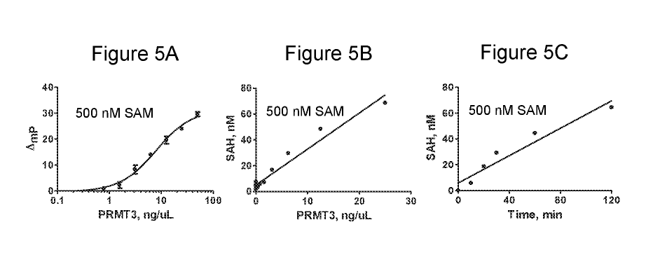

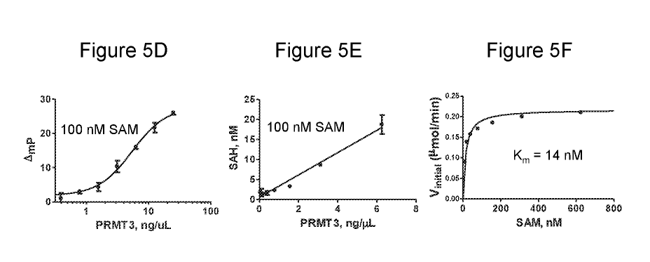

Based upon successes with the FP-based split aptamer format of Example 2, an assay for detection of HMT enzyme activity was next evaluated. First, a dose response was performed with the protein arginine HMT PRMT3 (UniProt Accession No. 060678; SEQ ID NO:9) in the presence of 500 nM SAM, equal to the Kₘ; an enzyme-dependent increase in polarization was observed (FIG. 5A). Because it relies on a saturable binding reaction, the response of the assay was hyperbolic rather than linear. However, when the polarization values were converted to the amount of SAH formed using a standard curve, the response was linear with enzyme concentration, as expected for an initial velocity reaction (FIG. 5B). The time dependence of the reaction was then demonstrated, and as shown in FIG. 5C, it was linear for at least 1 h, again reflecting initial velocity enzyme kinetics.

To demonstrate the utility of the assay at lower SAM concentrations, PRMT3 activity was measured at 100 nM SAM (FIG. 5D). Polarization increased about 25 mP between 1 and 20 ng/mL PRMT3. Conversion of this data to SAH formation, showed that the linear part of the response represented detection of SAH concentrations between 2 and 20 nM (FIG. 5E), which is well below the sensitivity of current assay methods. Additionally, from a plot of velocity vs. SAM, a Kₘ of 14 nM for the related HMT PRMT1 was determined (FIG. 5F). The magnitude of the polarization changes at these SAH concentrations (FIGS. 5D-5F) was not sufficient for HTS, but as is evident from the error bars, the assay was quite precise and these results clearly demonstrate the capability of the split aptamer assay to accurately measure enzyme activity at low SAM concentrations, which is critical for a significant number of HMTs. It should be noted that 100 nM SAM is less than one fourth of the minimum concentration that can be used with current HTS assay methods.

### Example 4: Development of a Novel SAH Biosensor with a Positive TR-FRET Signal

The split aptamer SAH detection assay was next formatted for TR-FRET readout. Luminescent lanthanides were attached to P1₅₂ of the SAH aptamer via streptavidin-biotin, and organic acceptor fluors were attached to P2₁₈ during synthesis. Both Tb and Eu lanthanide chelates (Life Technologies) were tested, as were four different organic fluors with excitation spectra overlapping the lanthanide emission (Alexa 633, Alexa 647, Cy5, and Dylight 650.)

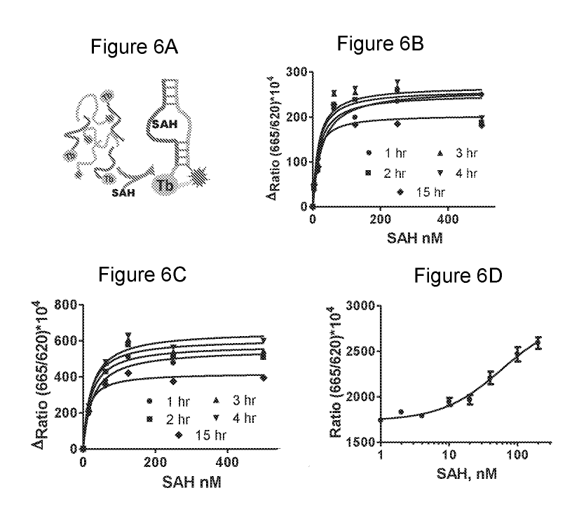

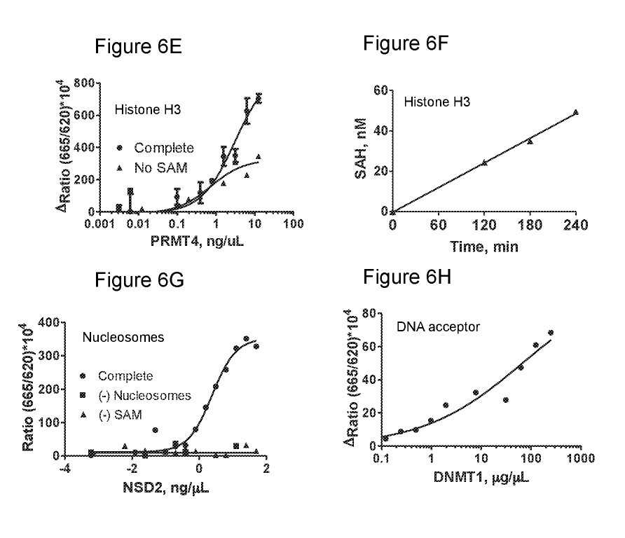

FRET increased significantly in a dose dependent manner as SAH was added (FIGS. 6B and 6C), indicating that the SAH-dependent association of P1₅₂ and P2₁₈ co-localized the lanthanide donors and acceptor fluors. Importantly, the signal was stable for at least 15 h. SAH EC₅₀ values of 23 and 16 nM for the Tb and Eu constructs, respectively, determined from the dose response curves in FIGS. 6B and 6C confirmed that high affinity binding was retained with the split aptamer TR-FRET configuration. There were minor differences in the FRET efficiencies with different acceptor fluors, but the Tb chelate was more effective that Eu with all of them; Tb/Dylight 650 was used for further studies. A standard curve for conversion of 200 nM SAM to SAH indicated that the TR-FRET based assay has the sensitivity and selectivity required for detecting HMT activity (FIG. 6D).

HMTs have diverse substrate requirements, which include short peptides, full length histones, and intact nucleosome complexes. Many enzymes will use more than one type of substrate, but some have a strict requirement for intact nucleosomes, including Dot1L and Nsd2 (Kumar et al., 2015, Assay Drug Dev. 13(4):200-9). Because nucleosomes are a heterogeneous, partially purified cell fraction, it was important to demonstrate compatibility with HMT assay methods. Accordingly, to test the TR-FRET assay for detection of enzyme activity, HMT PRMT4 (UniProt Accession No. Q86X55; SEQ ID NO:10) with full length histone H3 as substrate, HMT NSD2 (UniProt Accession No. 096028; SEQ ID NO:11) with oligonucleosomes, and DNA methyltransferase I (DNMT1; UniProt Accession No. P26358; SEQ ID NO:12) were used with a synthetic polynucleotide substrate (FIGS. 6E-6H); SAM concentrations of 200 nM (PRMT4) or 2 μM (NSD2 and DNMT1) were used for these assays. In all cases, dose dependent increases in the TR-FRET signal were observed as enzyme was added to the assay reagents, and linear SAH formation over time was demonstrated for PRMT4. Taken together with the FP data of Examples 2 and 3, these results clearly showed that the split aptamer based assay is compatible with the commonly used HMT and DNMT substrates and is useful for detection of diverse methyltransferase enzymes.

### Example 5: Optimization of Aptamer Binding for Detection of PTMs

Aptamers against the PTM targets of interest: 5′-AGACGTAAGTTAATTGGACTTGGTCGTGTGCGGCACAGCGATTGAAAT-3′ (SEQ ID N0:2) for the detection of Histone H4K16Ac and 5′-GAUGGGUCAGCAUGUAGCCAGGCAGGGCCGUGUGAGCUUGUGCUGAUGUG-3′ (SEQ ID NO:3) for the detection of Histone H3R8Me2sym are used. For initial evaluation of the aptamer binding parameters, modified peptides representing target sites are used, which are labeled with fluors to allow FP-based equilibrium binding analysis. Binding of the aptamers to the labeled peptides can produce a significant increase in polarization. The peptide SGRGKGGKGLGKGGAKacRHR (SEQ ID NO:13), representing H4K16Ac, and ARTKQTARme2symKSTGGKAPRKQ (SEQ ID NO:14), representing H3R8Me2sym, are synthesized with an AlexaFluor-633 at the C-terminus (Anaspec, Fremont, Calif.). 2-4 nM of the labeled peptides are incubated with varying amounts of will aptamer (0.1 nM to 1 μM), and changes in the FP signal are detected using BMG PHERAstar (Cary, N.C.). Because the buffer conditions including the type of salt, pH, and ionic strength can influence aptamer folding as well as binding kinetics to the target, these parameters are optimized to maximize aptamer/protein interaction as indicated by a greater FP shift and a lower K_{d} value. In addition, aptamer binding against full-length histone proteins containing or lacking the H4K16Ac and H3R8Me2sym modifications (available from Active Motif, Carlsbad, Calif.) is characterized.

### Example 6: Development of a Split Aptamer Pair that Reassembles in the Presence of the Target PTM

An iterative approach is used to generate split aptamer fragments, called P1 and P2, without compromising the binding specificity as compared to the full-length aptamer. Initial fragment-pairs are generated within the loop or near the center of non-folded region based on predicted structure (FIG. 4). To ensure that the split site does not perturb the aptamer's binding ability, 2-3 additional segments are also generated that are 5 bases upstream or downstream from the initial split point. Fluorescently labeled peptides with the target PTM are used to assess binding, and control reactions lacking P1 and P2 are used to confirm that the split aptamer reassembles to form a trimeric complex with the ligand. P1 and P2, present at an equimolar concentration of 2-4 nM, are incubated with 1-100 nM of the target protein for 1 h at room temperature, and changes in FP are measured. To maximize split aptamer binding to the target, buffer conditions are re-optimized as needed. The most promising split aptamers are fully characterized for affinity and specificity using modified peptides and full-length histones as well as the MODified™ Histone Peptide Array. Furthermore, the stability of the trimeric split aptamer/protein complex over a 24 h time-period is determined.

It is expected that the affinity and selectivity of the split aptamer is comparable to that of the full-length aptamer. Detection of at least 10 nM of target protein with a signal-to-background ratio >1:5 is also expected. In addition, K_{d} values are anticipated to be comparable to the full-length intact aptamer. The response is expected to reach 80% of maximal in less than 30 min. If difficulties are encountered, a short section of one or both aptamers will be selectively reengineered by modifying bases around the loop region that can facilitate their function as split aptamers. This general method involves removing a loop region and then systematically modifying the number of base pairs in the remaining stem region to achieve selective assembly only in the presence of the target, thereby providing splitting sites that are distal from the target-binding pocket, as described in Kent et al., 2013, Analytical Chemistry 85(20):9916-23.

### Example 7: Combining Split Aptamer with Enzyme Fragment Complementation Using Split Luciferase

Gene sequences encoding NLuc (residues 2-416; SEQ ID NO:15) and CLuc (residues 398-550; SEQ ID NO:16) fragments of firefly luciferase are used, and the two enzyme fragments are translated using a cell-free eukaryotic protein expression system—flexi-rabbit reticulocyle lysate (Promega, Madison, Wis.)—that has been successfully used to express split luciferase fusion protein constructs specifically for EFC-based detection (Porter et al., 2008, J Am. Chem. Soc. 130(20):6488-97 and Stains et al., 2010, ACS Chem. Bio. 5(10):943-52). The gene sequences for NLuc and CLuc are modified to include a terminal histidine (His) tag that is helpful for their downstream isolation and purification, and an overlapping 12 aa sequence to prevent luciferase self-association. Translations are carried out using 2 pmol of each split enzyme encoding RNA using the protocols described by the manufacturer. NLuc and CLuc fragments are purified using Promega's MagZ™ protein purification kit, which can achieve 99.9% purification of His-tagged proteins. The concentration of the purified NLuc and CLuc proteins are determined using the BCA assay.

While the role of the His-tag on NLuc and CLuc fragments is primarily for purification, their presence is used in order to conjugate each enzyme fragment to each split aptamer fragment separately. The 5′-end of P1 and the 3′-end of P2 is modified with nitrilotriacetate (NTA) via thiol linkage (Gene Link, Hawthrome, N.Y.). NTA-modified oligos have high affinity towards His-tag due to metal affinity complexation in presence of Ni²⁺ (Geissler, D., et al., 2014, Inorg Chem, 53(4):1824-38; Wegner, K. D., et al., 2013, ACS Applied Materials & Interfaces, 5(8):2881-2892; Harma, H., et al., 2007, Anal Chim Acta, 604(2):177-83) and have been previously used to conjugate aptamers to proteins (Wegner, K. D., et al., 2013, ACS Nano, 7(8):7411-9; Tanaka, S., et al., 2003, Biophysical Journal, 84(5):3299-3306). The level of background signal, if any, from the undesirable interaction of NLuc/CLuc fragments or NLuc-P1 fusion/CLuc-P2 fusion is determined by measuring the luminescence of the sample in a M1000-Pro multi-mode plate reader (Tecan, Männedorf, Switzerland). To test PTM recognition, 10-100 nM of the split aptamer probes are incubated with 0.1 nM to 10 μM of the target at room temperature for 1 h. The luminescence resulting from split aptamer driven co-assembly of NLuc and CLuc fragments, and hence luciferase activity restoration, is measured and expressed as a ratio of signal to background. To test for specificity, the probes are incubated with several non-target PTMs and probe signal is measured. The K_{d} and response time are calculated and compared to that of the intact aptamer.

Detection of the target PTM protein at a sensitivity of at least 10 nM, a signal-to-background ratio of >5, and a response time <30 min is expected. It is also expected that the split aptamer probes demonstrate >50-fold higher specificity towards non-target PTM molecules. If a low dynamic range or high background signal are encountered, the aptamer/enzyme attachment sequence are reversed and both configurations are tested.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

TABLE 1
Sequences disclosed herein.

SEQ ID NO: 1

| | |
|---|---|
| gggucuucca aggagcguug cagucggcca cauggccggu caggcuugga ugaccccaac | 60 |
| gacgcucacc tgauccauuu agcuacaggu gaguugca | 98 |

SEQ ID NO: 2

| | |
|---|---|
| agacgtaagt taattggact tggtcgtgtg cggcacagcg attgaaat | 48 |

SEQ ID NO: 3

| | |
|---|---|
| gaugggucag cauguagcca ggcagggccg ugugagcuug ugcugaugug | 50 |

SEQ ID NO: 4

| | |
|---|---|
| gggucugccg aggagcgcug cgacccuuua auucgggggc caggcucggc aaugaucaac | 60 |
| ggcgcucgcc ggc | 73 |

SEQ ID NO: 5

| | |
|---|---|
| CY5-gagcgccguu | 10 |

SEQ ID NO: 6

| | |
|---|---|
| gggucugccg aggagcgcug cgacccuuua auucgggggc caggcucggc aaugaugcc | 59 |

SEQ ID NO: 7

| | |
|---|---|
| ccgaggagcg cugcgacccu uuaauucggg ggccaggcuc ggcaaugaug cc | 52 |

SEQ ID NO: 8

| | |
|---|---|
| augaucaacg gcgcucgc | 18 |

SEQ ID NO: 9

| | |
|---|---|
| MCSLASGATG GRGAVENEED LPELSDSGDE AAWEDEDDAD LPHGKQQTPC LFCNRLFTSA | 60 |
| EETFSHCKSE HQFNIDSMVH KHGLEFYGYI KLINFIRLKN PTVEYMNSIY NPVPWEKEEY | 120 |
| LKPVLEDDLL LQFDVEDLYE PVSVPFSYPN GLSENTSVVE KLKHMEARAL SAEAALARAR | 180 |
| EDLQKMKQFA QDFVMHTDVR TCSSSTSVIA DLQEDEDGVY FSSYGHYGIH EEMLKDKIRT | 240 |
| ESYRDFIYQN PHIFKDKVVL DVGCGTGILS MFAAKAGAKK VLGVDQSEIL YQAMDIIRLN | 300 |
| KLEDTITLIK GKIEEVHLPV EKVDVIISEW MGYFLLFESM LDSVLYAKNK YLAKGGSVYP | 360 |
| DICTISLVAV SDVNKHADRI AFWDDVYGFK MSCMKKAVIP EAVVEVLDPK TLISEPCGIK | 420 |
| HIDCHTTSIS DLEFSSDFTL KITRISMCIA IAGYFDIYFE KNCHNRVVFS TGPQSTKTHW | 480 |
| KQTVFLLEKP FSVKAGEALK GKVTVHKSKK DPRSLTVTLT LNNSTQTYGL Q | 531 |

SEQ ID NO: 10

| | |
|---|---|
| MAAAAAAVGP GAGGAGSAVP GGAGPCATVS VFPGARLLTI GDANGEIQRH AEQQALRLEV | 60 |
| RAGPDSAGIA LYSHEDVCVF KCSVSRETEC SRVGKQSFII TLGCNSVLIQ FATPNDFCSF | 120 |
| YNILKTCRGH TLERSVFSER TEESSAVQYF QFYGYLSQQQ NMMQDYVRTG TYQRAILQNH | 180 |
| TDFKDKIVLD VGCGSGILSF FAAQAGARKI YAVEASTMAQ HAEVLVKSNN LTDRIVVIPG | 240 |
| KVEEVSLPEQ VDIIISEPMG YMLFNERMLE SYLHAKKYLK PSGNMFPTIG DVHLAPFTDE | 300 |
| QLYMEQFTKA NFWYQPSFHG VDLSALRGAA VDEYFRQPVV DTFDIRILMA KSVKYTVNFL | 360 |
| EAKEGDLHRI EIPFKFHMLH SGLVHGLAFW FDVAFIGSIM TVWLSTAPTE PLTHWYQVRC | 420 |
| LFQSPLFAKA GDTLSGTCLL IANKRQSYDI SIVAQVDQTG SKSSNLLDLK NPFFRYTGTT | 480 |
| PSPPPGSHYT SPSENMWNTG STYNLSSGMA VAGMPTAYDL SSVIASGSSV GHNNLIPLAN | 540 |
| TGIVNHTHSR MGSIMSTGIV QGSSGAQGSG GGSTSAHYAV NSQFTMGGPA ISMASPMSIP | 600 |
| TNTMHYGS | 608 |

SEQ ID NO: 11

| | |
|---|---|
| MEFSIKQSPL SVQSVVKCIK MKQAPEILGS ANGKTPSCEV NRECSVFLSK AQLSSSLQEG | 60 |
| VMQKFNGHDA LPFIPADKLK DLTSRVFNGE PGAHDAKLRF ESQEMKGIGT PPNTTPIKNG | 120 |
| SPEIKLKITK TYMNGKPLFE SSICGDSAAD VSQSEENGQK PENKARRNRK RSIKYDSLLE | 180 |
| QGLVEAALVS KISSPSDKKI PAKKESCPNT GRDKDHLLKY NVGDLVWSKV SGYPWWPCMV | 240 |
| SADPLLHSYT KLKGQKKSAR QYHVQFFGDA PERAWIFEKS LVAFEGEGQF EKLCQESAKQ | 300 |
| APTKAEKIKL LKPISGKLRA QWEMGIVQAE EAASMSVEER KAKFTFLYVG DQLHLNPQVA | 360 |
| KEAGIAAESL GEMAESSGVS EEAAENPKSV REECIPMKRR RRAKLCSSAE TLESHPDIGK | 420 |
| STPQKTAEAD PRRGVGSPPG RKKTTVSMPR SRKGDAASQF LVFCQKHRDE VVAEHPDASG | 480 |
| EEIEELLRSQ WSLLSEKQRA RYNTKFALVA PVQAEEDSGN VNGKKRNHTK RIQDPTEDAE | 540 |
| AEDTPRKRLR TDKHSLRKRD TITDKTARTS SYKAMEAASS LKSQAATKNL SDACKPLKKR | 600 |
| NRASTAASSA LGFSKSSSPS ASLTENEVSD SPGDEPSESP YESADETQTE VSVSSKKSER | 660 |
| GVTAKKEYVC QLCEKPGSLL LCEGPCCGAF HLACLGLSRR PEGRFTCSEC ASGIHSCFVC | 720 |
| KESKTDVKRC VVTQCGKFYH EACVKKYPLT VFESRGFRCP LHSCVSCHAS NPSNPRPSKG | 780 |
| KMMRCVRCPV AYHSGDACLA AGCSVIASNS IICTAHFTAR KGKRHHAHVN VSWCFVCSKG | 840 |
| GSLLCCESCP AAFHPDCLNI EMPDGSWFCN DCRAGKKLHF QDIIWVKLGN YRWWPAEVCH | 900 |
| PKNVPPNIQK MKHEIGEFPV FFFGSKDYYW THQARVFPYM EGDRGSRYQG VRGIGRVFKN | 960 |
| ALQEAEARFR EIKLQREARE TQESERKPPP YKHIKVNKPY GKVQIYTADI SEIPKCNCKP | 1020 |
| TDENPCGFDS ECLNRMLMFE CHPQVCPAGE FCQNQCFTKR QYPETKIIKT DGKGWGLVAK | 1080 |
| RDIRKGEFVN EYVGELIDEE ECMARIKHAH ENDITHFYML TIDKDRIIDA GPKGNYSRFM | 1140 |
| NHSCQPNCET LKWTVNGDTR VGLFAVCDIP AGTELTFNYN LDCLGNEKTV CRCGASNCSG | 1200 |
| FLGDRPKTST TLSSEEKGKK TKKKTRRRRA KGEGKRQSED ECFRCGDGGQ LVLCDRKFCT | 1260 |
| KAYHLSCLGL GKRPFGKWEC PWHHCDVCGK PSTSFCHLCP NSFCKEHQDG TAFSCTPDGR | 1320 |
| SYCCEHDLGA ASVRSTKTEK PPPEPGKPKG KRRRRRGWRR VTEGK | 1365 |

SEQ ID NO: 12

| | |
|---|---|
| MPARTAPARV PTLAVPAISL PDDVRRRLKD LERDSLTEKE CVKEKLNLLH EFLQTEIKNQ | 60 |
| LCDLETKLRK EELSEEGYLA KVKSLLNKDL SLENGAHAYN REVNGRLENG NQARSEARRV | 120 |
| GMADANSPPK PLSKPRTPRR SKSDGEAKPE PSPSPRITRK STRQTTITSH FAKGPAKRKP | 180 |
| QEESERAKSD ESIKEEDKDQ DEKRRRVTSR ERVARPLPAE EPERAKSGTR TEKEEERDEK | 240 |
| EEKRLRSQTK EPTPKQKLKE EPDREARAGV QADEDEDGDE KDEKKHRSQP KDLAAKRRPE | 300 |
| EKEPEKVNPQ ISDEKDEDEK EEKRRKTTPK EPTEKKMARA KTVMNSKTHP PKCIQCGQYL | 360 |
| DDPDLKYGQH PPDAVDEPQM LTNEKLSIFD ANESGFESYE ALPQHKLTCF SVYCKHGHLC | 420 |
| PIDTGLIEKN IELFFSGSAK PIYDDDPSLE GGVNGKNLGP INEWWITGFD GGEKALIGFS | 480 |
| TSFAEYILMD PSPEYAPIFG LMQEKIYISK IVVEFLQSNS DSTYEDLINK IETTVPPSGL | 540 |
| NLNRFTEDSL LRHAQFVVEQ VESYDEAGDS DEQPIFLTPC MRDLIKLAGV TLGQRRAQAR | 600 |
| RQTIRHSTRE KDRGPTKATT TKLVYQIFDT FFAEQIEKDD REDKENAFKR RRCGVCEVCQ | 660 |
| QPECGKCKAC KDMVKFGGSG RSKQACQERR CPNMAMKEAD DDEEVDDNIP EMPSPKKMHQ | 720 |
| GKKKKQNKNR ISWVGEAVKT DGKKSYYKKV CIDAETLEVG DCVSVIPDDS SKPLYLARVT | 780 |
| ALWEDSSNGQ MFHAHWFCAG TDTVLGATSD PLELFLVDEC EDMQLSYIHS KVKVIYKAPS | 840 |
| ENWAMEGGMD PESLLEGDDG KTYFYQLWYD QDYARFESPP KTQPTEDNKF KFCVSCARLA | 900 |
| EMRQKEIPRV LEQLEDLDSR VLYYSATKNG ILYRVGDGVY LPPEAFTFNI KLSSPVKRPR | 960 |
| KEPVDEDLYP EHYRKYSDYI KGSNLDAPEP YRIGRIKEIF CPKKSNGRPN ETDIKIRVNK | 1020 |
| FYRPENTHKS TPASYHADIN LLYWSDEEAV VDFKAVQGRC TVEYGEDLPE CVQVYSMGGP | 1080 |
| NRFYFLEAYN AKSKSFEDPP NHARSPGNKG KGKGKGKGKP KSQACEPSEP EIEIKLPKLR | 1140 |
| TLDVFSGCGG LSEGFHQAGI SDTLWAIEMW DPAAQAFRLN NPGSTVFTED CNILLKLVMA | 1200 |
| GETTNSRGQR LPQKGDVEML CGGPPCQGFS GMNRFNSRTY SKFKNSLVVS FLSYCDYYRP | 1260 |
| RFFLLENVRN FVSFKRSMVL KLTLRCLVRM GYQCTFGVLQ AGQYGVAQTR RRAIILAAAP | 1320 |
| GEKLPLFPEP LHVFAPRACQ LSVVVDDKKF VSNITRLSSG PFRTITVRDT MSDLPEVRNG | 1380 |
| ASALEISYNG EPQSWFQRQL RGAQYQPILR DHICKDMSAL VAARMRHIPL APGSDWRDLP | 1440 |
| NIEVRLSDGT MARKLRYTHH DRKNGRSSSG ALRGVCSCVE AGKACDPAAR QFNTLIPWCL | 1500 |
| PHTGNRHNHW AGLYGRLEWD GFFSTTVTNP EPMGKQGRVL HPEQHRVVSV RECARSQGFP | 1560 |
| DTYRLFGNIL DKHRQVGNAV PPPLAKAIGL EIKLCMLAKA RESASAKIKE EEAAKD | 1616 |

SEQ ID NO: 13

SGRGKGGKGLGKGGAKacRHR

SEQ ID NO: 14

ARTKQTARme2symKSTGGKAPRKQ

SEQ ID NO: 15

| | |
|---|---|
| MEDAKNIKKG PAPFYPLEDG TAGEQLHKAM KRYALVPGTI AFTDAHIEVN ITYAEYFEMS | 60 |
| VRLAEAMKRY GLNTNHRIVV CSENSLQFFM PVLGALFIGV AVAPANDIYN ERELLNSMNI | 120 |
| SQPTVVFVSK KGLQKILNVQ KKLPIIQKII IMDSKTDYQG FQSMYTFVTS HLPPGFNEYD | 180 |
| FVPESFDRDK TIALIMNSSG STGLPKGVAL PHRTACVRFS HARDPIFGNQ IIPDTAILSV | 240 |
| VPFHHGFGMF TTLGYLICGF RVVLMYRFEE ELFLRSLQDY KIQSALLVPT LFSFFAKSTL | 300 |
| IDKYDLSNLH EIASGGAPLS KEVGEAVAKR FHLPGIRQGY GLTETTSAIL ITPEGDDKPG | 360 |
| AVGKVVPFFE AKVVDLDTGK TLGVNQRGEL CVRGPMIMSG YVNNPEATNA LIDKDGGGGS | 420 |
| SGGGQISYAS RGHHHHHH | 438 |

SEQ ID NO: 16

| | |
|---|---|
| MASGYVNNPE ATNALIDKDG WLHSGDIAYW DEDEHFFIVD RLKSLIKYKG YQVAPAELES | 60 |
| ILLQHPNIFD AGVAGLPDDD AGELPAAVVV LEHGKTMTEK EIVDYVASQV TTAKKLRGGV | 120 |
| VFVDEVPKGL TGKLDARKIR EILIKAKKGG KSKLGGGSSG GGQISYASRG HHHHHH | 176 |

What is claimed is:

1. A sensor for measuring an analyte, comprising:
(a) a first fragment of a split aptamer and
(b) a second fragment of the split aptamer;
wherein the first fragment of the split aptamer comprises a first modification;
wherein the first fragment of the split aptamer and the second fragment of the split aptamer are associated in the presence of the analyte to form a trimeric complex with the analyte.

2. The sensor of claim 1, wherein the first fragment of the split aptamer and the second fragment of the split aptamer are DNA and/or RNA molecules.

3. The sensor of claim 2, wherein the DNA and/or RNA molecules comprise modified nucleotides.

4. The sensor of claim 1, wherein the first modification is a fluor modification.

5. The sensor of claim 4, wherein the fluor modification is a fluorescein, rhodamine, texas red, an alexa fluor, a cyanine dye, or an atto dye modification.

6. The sensor of claim 4 or claim 5, wherein the fluor modification is attached at a terminus of the split aptamer or internally in the split aptamer.

7. The sensor of claim 1, wherein the first modification is a streptavidin modification.

8. The sensor of any one of claims 1-7, wherein a measured fluorescence polarization (FP) induced by the trimeric complex is larger than a measured FP induced by the first fragment of the split aptamer and the second fragment of the split aptamer prior to assembly of the trimeric complex.

9. The sensor of any one of claims 1-7, wherein the second fragment of the split aptamer further comprises a second modification.

10. The sensor of claim 9, wherein the second modification is a luminescent lanthanide modification.

11. The sensor of claim 10, wherein the luminescent lanthanide is terbium or europium.

12. The sensor of claim 9, wherein the second modification is an upconversion nanoparticle.

13. The sensor of any one of claims 1-12, wherein the trimeric complex produces a time-resolved fluorescence energy transfer (TR-FRET) signal.

14. A sensor for measuring an analyte, comprising:
(a) a first fragment of a split aptamer and
(b) a second fragment of a split aptamer;
wherein the first fragment of the split aptamer is conjugated to a first fragment of a reporter enzyme polypeptide;
wherein the second fragment of the split aptamer is conjugated to a second fragment of a reporter enzyme polypeptide;
wherein the first fragment of the split aptamer and the second fragment of the split aptamer are associated in the presence of the analyte to form a trimeric complex with the analyte.

15. The sensor of claim 14, wherein the first fragment of the split aptamer and the second fragment of the split aptamer are DNA and/or RNA molecules.

16. The sensor of claim 15, wherein the DNA and/or RNA molecules comprise modified nucleotides.

17. The sensor of any one of claim 14-16, wherein the first fragment of the reporter enzyme polypeptide and the second fragment of the reporter enzyme polypeptide are complementary fragments of a split reporter enzyme.

18. The sensor of any one of claims 14-17, wherein the first fragment of the reporter enzyme polypeptide and the second fragment of the reporter enzyme polypeptide assemble into an intact reporter enzyme in the presence of the analyte.

19. The sensor of claim 18, wherein the intact reporter enzyme is a luciferase protein.

20. The sensor of any one of claims 14-19, wherein the first fragment of the reporter enzyme polypeptide has at least 90% identity to the amino acid sequence set forth in SEQ ID NO:15 and the second fragment of the reporter enzyme polypeptide has at least 90% identity to the amino acid sequence set forth in SEQ ID NO:16.

21. The sensor of claim 19, wherein the luciferase protein produces a luminescent signal upon conversion of a luciferin substrate.

22. The sensor of any one of claims 1-21, wherein the analyte is an amino acid, an amino acid-related molecule, a peptide, a protein, a steroid, a lipid, a sugar, a carbohydrate, a drug molecule, a drug metabolite, a coenzyme, a nucleotide, a nucleotide-related molecule, a pyridine nucleotide, a cyclic nucleotide, or a cyclic dinucleotide.

23. The sensor of claim 22, wherein the analyte is S-adenosylhomocysteine (SAH).

24. The sensor of claim 22, wherein the analyte is a protein having a post-translational modification (PTM).

25. The sensor of claim 24, wherein the analyte is an acetylated and/or methylated histone.

26. A method for detecting an analyte, comprising:
(a) contacting the sensor of any one of claims 1-25 with a sample;
wherein the first fragment of the split aptamer and the second fragment of the split aptamer assemble in the presence of the analyte to form the trimeric complex with the analyte; and
(b) measuring a signal generated upon assembly of the trimeric complex.

27. The method of claim 26, wherein the signal generated is measured by FP, TR-FRET, and/or luminescence.

28. The method of claim 26 or claim 27, wherein the analyte is detected in a high-throughput screen (HTS).


SEQUENCE LISTING (16 sequences)

SEQ ID NO: 1 - DNA, 98 bp, Rhodoferax ferrireducens
gggucuuccaaggagcguugcagucggccacauggccggucaggcuuggaugaccccaacgacgcucacctgauccauuuagcuacaggugaguugca

SEQ ID NO: 2 - DNA, 48 bp, Artificial Sequence
  H4K16Ac aptamer
agacgtaagttaattggacttggtcgtgtgcggcacagcgattgaaat

SEQ ID NO: 3 - RNA, 50 nt, Artificial Sequence
  H3R8Me2sym aptamer
gaugggucagcauguagccaggcagggccgugugagcuugugcugaugug

SEQ ID NO: 4 - RNA, 73 nt, Artificial Sequence
  Dar1 aptamer
gggucugccgaggagcgcugcgacccuuuaauucgggggccaggcucggcaaugaucaacggcgcucgccggc

SEQ ID NO: 5 - RNA, 10 nt, Artificial Sequence
  Synthetic oligo
  misc_feature (1)..(1): Cy5
gagcgccguu

SEQ ID NO: 6 - RNA, 59 nt, Artificial Sequence
  P1_59 5′ element
gggucugccgaggagcgcugcgacccuuuaauucgggggccaggcucggcaaugaugcc

SEQ ID NO: 7 - RNA, 52 nt, Artificial Sequence
  P1_52 5′ element
ccgaggagcgcugcgacccuuuaauucgggggccaggcucggcaaugaugcc

SEQ ID NO: 8 - RNA, 18 nt, Artificial Sequence
  P2_18 3′ element
augaucaacggcgcucgc

SEQ ID NO: 9 - protein, 531 aa, Homo sapiens
MCSLASGATGGRGAVENEEDLPELSDSGDEAAWEDEDDADLPHGKQQTPCLFCNRLFTSAEETFSHCKSEHQFNIDSMVHKHGLEFYGYIKLINFIRLKNPTVEYMNSIYNPVPWEKEEYLKPVLEDDLLLQFDVEDLYEPVSVPFSYPNGLSENTSVVEKLKHMEARALSAEAALARAREDLQKMKQFAQDFVMHTDVRTCSSSTSVIADLQEDEDGVYFSSYGHYGIHEEMLKDKIRTESYRDFIYQNPHIFKDKVVLDVGCGTGILSMFAAKAGAKKVLGVDQSEILYQAMDIIRLNKLEDTITLIKGKIEEVHLPVEKVDVIISEWMGYFLLFESMLDSVLYAKNKYLAKGGSVYPDICTISLVAVSDVNKHADRIAFWDDVYGFKMSCMKKAVIPEAVVEVLDPKTLISEPCGIKHIDCHTTSISDLEFSSDFTLKITRTSMCTAIAGYFDIYFEKNCHNRVVFSTGPQSTKTHWKQTVFLLEKPFSVKAGEALKGKVTVHKSKKDPRSLTVTLTLNNSTQTYGLQ

SEQ ID NO: 10 - protein, 608 aa, Homo sapiens
MAAAAAAVGPGAGGAGSAVPGGAGPCATVSVFPGARLLTIGDANGEIQRHAEQQALRLEVRAGPDSAGIALYSHEDVCVFKCSVSRETECSRVGKQSFIITLGCNSVLIQFATPNDFCSFYNILKTCRGHTLERSVFSERTEESSAVQYFQFYGYLSQQQNMMQDYVRTGTYQRAILQNHTDFKDKIVLDVGCGSGILSFFAAQAGARKIYAVEASTMAQHAEVLVKSNNLTDRIVVIPGKVEEVSLPEQVDIIISEPMGYMLFNERMLESYLHAKKYLKPSGNMFPTIGDVHLAPFTDEQLYMEQFTKANFWYQPSFHGVDLSALRGAAVDEYFRQPVVDTFDIRILMAKSVKYTVNFLEAKEGDLHRIEIPFKFHMLHSGLVHGLAFWFDVAFIGSIMTVWLSTAPTEPLTHWYQVRCLFQSPLFAKAGDTLSGTCLLIANKRQSYDISIVAQVDQTGSKSSNLLDLKNPFFRYTGTTPSPPPGSHYTSPSENMWNTGSTYNLSSGMAVAGMPTAYDLSSVIASGSSVGHNNLIPLANTGIVNHTHSRMGSIMSTGIVQGSSGAQGSGGGSTSAHYAVNSQFTMGGPAISMASPMSIPTNTMHYGS

SEQ ID NO: 11 - protein, 1365 aa, Homo sapiens
MEFSIKQSPLSVQSVVKCIKMKQAPEILGSANGKTPSCEVNRECSVFLSKAQLSSSLQEGVMQKFNGHDALPFIPADKLKDLTSRVFNGEPGAHDAKLRFESQEMKGIGTPPNTTPIKNGSPEIKLKITKTYMNGKPLFESSICGDSAADVSQSEENGQKPENKARRNRKRSIKYDSLLEQGLVEAALVSKISSPSDKKIPAKKESCPNTGRDKDHLLKYNVGDLVWSKVSGYPWWPCMVSADPLLHSYTKLKGQKKSARQYHVQFFGDAPERAWIFEKSLVAFEGEGQFEKLCQESAKQAPTKAEKIKLLKPISGKLRAQWEMGIVQAEEAASMSVEERKAKFTFLYVGDQLHLNPQVAKEAGIAAESLGEMAESSGVSEEAAENPKSVREECIPMKRRRRAKLCSSAETLESHPDIGKSTPQKTAEADPRRGVGSPPGRKKTTVSMPRSRKGDAASQFLVFCQKHRDEVVAEHPDASGEEIEELLRSQWSLLSEKQRARYNTKFALVAPVQAEEDSGNVNGKKRNHTKRIQDPTEDAEAEDTPRKRLRTDKHSLRKRDTITDKTARTSSYKAMEAASSLKSQAATKNLSDACKPLKKRNRASTAASSALGFSKSSSPSASLTENEVSDSPGDEPSESPYESADETQTEVSVSSKKSERGVTAKKEYVCQLCEKPGSLLLCEGPCCGAFHLACLGLSRRPEGRFTCSECASGIHSCFVCKESKTDVKRCVVTQCGKFYHEACVKKYPLTVFESRGFRCPLHSCVSCHASNPSNPRPSKGKMMRCVRCPVAYHSGDACLAAGCSVIASNSIICTAHFTARKGKRHHAHVNVSWCFVCSKGGSLLCCESCPAAFHPDCLNIEMPDGSWFCNDCRAGKKLHFQDIIWVKLGNYRWWPAEVCHPKNVPPNIQKMKHEIGEFPVFFFGSKDYYWTHQARVFPYMEGDRGSRYQGVRGIGRVFKNALQEAEARFREIKLQREARETQESERKPPPYKHIKVNKPYGKVQIYTADISEIPKCNCKPTDENPCGFDSECLNRMLMFECHPQVCPAGEFCQNQCFTKRQYPETKIIKTDGKGWGLVAKRDIRKGEFVNEYVGELIDEEECMARIKHAHENDITHFYMLTIDKDRIIDAGPKGNYSRFMNHSCQPNCETLKWTVNGDTRVGLFAVCDIPAGTELTFNYNLDCLGNEKTVCRCGASNCSGFLGDRPKTSTTLSSEEKGKKTKKKTRRRRAKGEGKRQSEDECFRCGDGGQLVLCDRKFCTKAYHLSCLGLGKRPFGKWECPWHHCDVCGKPSTSFCHLCPNSFCKEHQDGTAFSCTPDGRSYCCEHDLGAASVRSTKTEKPPPEPGKPKGKRRRRRGWRRVTEGK

SEQ ID NO: 12 - protein, 1616 aa, Homo sapiens
MPARTAPARVPTLAVPAISLPDDVRRRLKDLERDSLTEKECVKEKLNLLHEFLQTEIKNQLCDLETKLRKEELSEEGYLAKVKSLLNKDLSLENGAHAYNREVNGRLENGNQARSEARRVGMADANSPPKPLSKPRTPRRSKSDGEAKPEPSPSPRITRKSTRQTTITSHFAKGPAKRKPQEESERAKSDESIKEEDKDQDEKRRRVTSRERVARPLPAEEPERAKSGTRTEKEEERDEKEEKRLRSQTKEPTPKQKLKEEPDREARAGVQADEDEDGDEKDEKKHRSQPKDLAAKRRPEEKEPEKVNPQISDEKDEDEKEEKRRKTTPKEPTEKKMARAKTVMNSKTHPPKCIQCGQYLDDPDLKYGQHPPDAVDEPQMLTNEKLSIFDANESGFESYEALPQHKLTCFSVYCKHGHLCPIDTGLIEKNIELFFSGSAKPIYDDDPSLEGGVNGKNLGPINEWWITGFDGGEKALIGFSTSFAEYILMDPSPEYAPIFGLMQEKIYISKIVVEFLQSNSDSTYEDLINKIETTVPPSGLNLNRFTEDSLLRHAQFVVEQVESYDEAGDSDEQPIFLTPCMRDLIKLAGVTLGQRRAQARRQTIRHSTREKDRGPTKATTTKLVYQIFDTFFAEQIEKDDREDKENAFKRRRCGVCEVCQQPECGKCKACKDMVKFGGSGRSKQACQERRCPNMAMKEADDDEEVDDNIPEMPSPKKMHQGKKKKQNKNRISWVGEAVKTDGKKSYYKKVCIDAETLEVGDCVSVIPDDSSKPLYLARVTALWEDSSNGQMFHAHWFCAGTDTVLGATSDPLELFLVDECEDMQLSYIHSKVKVIYKAPSENWAMEGGMDPESLLEGDDGKTYFYQLWYDQDYARFESPPKTQPTEDNKFKFCVSCARLAEMRQKEIPRVLEQLEDLDSRVLYYSATKNGILYRVGDGVYLPPEAFTFNIKLSSPVKRPRKEPVDEDLYPEHYRKYSDYIKGSNLDAPEPYRIGRIKEIFCPKKSNGRPNETDIKIRVNKFYRPENTHKSTPASYHADINLLYWSDEEAVVDFKAVQGRCTVEYGEDLPECVQVYSMGGPNRFYFLEAYNAKSKSFEDPPNHARSPGNKGKGKGKGKGKPKSQACEPSEPEIEIKLPKLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKD

SEQ ID NO: 13 - protein, 19 aa, Artificial Sequence
  H4K16Ac peptide
  MOD_RES (16)..(16): ACETYLATION
SGRGKGGKGLGKGGAKRHR

SEQ ID NO: 14 - protein, 19 aa, Artificial Sequence
  H3R8Me2sym peptide
  METHYLATION (8)..(8): Symmetrical dimethylated arginine
ARTKQTARKSTGGKAPRKQ

SEQ ID NO: 15 - protein, 438 aa, Artificial Sequence
  Firefly luciferase fragment
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGGGGSSGGGQISYASRGHHHHHH

SEQ ID NO: 16 - protein, 176 aa, Artificial Sequence
  Firefly luciferase fragment
MASGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKLGGGSSGGGQISYASRGHHHHHH